(19) United States
(12) Patent Application Publication
(10) Pub. No.: US 2013/0072804 A1
(43) Pub. Date: Mar. 21, 2013

(54) LIGHT TRANSMITTING PROBE, LIGHT RECEIVING PROBE, LIGHT TRANSMITTING AND RECEIVING PROBE, AND LIGHT MEASUREMENT DEVICE USING SAME

(71) Applicant: Yoshihiro Inoue, Kyoto (JP)

(72) Inventor: Yoshihiro Inoue, Kyoto (JP)

(73) Assignee: SHIMADZU CORPORATION, Kyoto (JP)

(21) Appl. No.: 13/701,396

(22) PCT Filed: Aug. 31, 2010
(86) PCT No.: PCT/JP2010/064779

§ 371 Date: Nov. 30, 2012

(51) Int. Cl. A61B 5/00

(52) U.S. Cl. CPC A61B 5/0082
USPC 600/476

(57) ABSTRACT

A light transmitting probe comprising: a housing for fixing the probe to a mounting portion of a holder to be mounted on a subject; a light emitter for emitting light, the light emitter being placed in an end portion of the housing; and a transmission channel, one end of which is connected to the light emitter, and the other end of each is connected to a controller; the light transmitting probe irradiating the subject with light when fixed to the holder, and being characterized in that the end portion of the housing has a number of rod-shaped protrusions, the light emitter is a number of light emitting elements, the light emitting elements are respectively placed in an end portion of each protrusion, the transmission channel is a number of transmission channels, and the transmission channels are respectively placed inside each protrusion.

### TECHNICAL FIELD

The present invention relates to a light transmitting probe, a light receiving probe, a light transmitting and receiving probe, and a light measurement device using the same for noninvasively measuring brain activity using light.

### BACKGROUND ART

In recent years, optical brain function imaging devices (light measurement devices) for simple and noninvasive measurement using light have been developed in order to observe brain activity. These optical brain function imaging devices are provided with light transmitting probes and light receiving probes. As a result, the optical brain function imaging devices irradiate a brain with near-infrared rays having three different wavelengths λ₁, λ₂ and λ₃ (780 nm, 805 nm and 830 nm, for example) from light transmitting probes placed on the surface of the head of a subject, and at the same time detect the respective intensities of near-infrared rays having the respective wavelengths that have been released from the brain (information on the amounts of received light) A(λ₁), A(λ₂) and A(λ₃) by means of the light receiving probes placed on the surface of the head.

In order to find the product [oxyHb] of the concentration of oxyhemoglobin in the cerebral blood flow and the length of the light path as well as the product [deoxyHb] of the concentration of deoxyhemoglobin and the length of the light path from the thus-gained information on the amounts of received light A(λ₁), A(λ₂) and A(λ₃), simultaneous equations shown as the following relational expressions (1), (2) and (3) are prepared using the Modified Beer Lambert Law, for example, and the simultaneous equations are solved (see Non-Patent Document 1). Furthermore, the product of the concentration of the total hemoglobin and the length of the light path ([oxyHb]+[deoxyHb]) is calculated from the product [oxyHb] of the concentration of oxyhemoglobin and the length of the light path as well as the product [deoxyHb] of the concentration of deoxyhemoglobin and the length of the light path.

A(λ₁)=E_{O}(λ₁)×[oxyHb]+E_{d}(λ₁)×[deoxyHb] (1)

A(λ₂)=E_{O}(λ₂)×[oxyHb]+E_{d}(λ₂)×[deoxyHb] (2)

A(λ₃)=E_{O}(λ₃)×[oxyHb]+E_{d}(λ₃)×[deoxyHb] (3)

Here, E_{O}(λm) is the coefficient of light absorption by the oxyhemoglobin when light has a wavelength λm, and E_{d}(λm) is the coefficient of light absorption by the deoxyhemoglobin when light has a wavelength λm.

Here, the relationship between the portion to be measured and the distance (channel) between the light transmitting probe and the light receiving probe is described. FIG. 5(a) is a cross-sectional diagram showing the relationship between a portion to be measured and a pair of probes, light transmitting probe and light receiving probe, and FIG. 5(b) is a plan diagram of FIG. 5(a).

The light transmitting probe 112 is pressed against the light transmitting point T on the surface of the head of the subject, and at the same time, the light receiving probe 113 is pressed against the light receiving point R on the surface of the head of the subject. Thus, light is emitted from the light transmitting probe 112, and at the same time, the light released from the surface of the head enters into the light receiving probe 113. At this time, the light that has been emitted through the light transmitting point T on the surface of the head and passed through the banana-shaped area (area to be measured) reaches the light receiving point R on the surface of the head. As a result, information on the amounts of received light A(λ₁), A(λ₂) and A(λ₃) for the particular portion to be measured S of the subject that is at the depth L/2, which is half of the length of the line segment L directly connecting the light transmitting point T and the light receiving point R along the surface of the head of the subject, from the middle point M of the line segment L can be obtained from the region to be measured.

In order to measure the product [oxyHb] of the concentration of the oxyhemoglobin and the length of the light path, the product [deoxyHb] of the concentration of the deoxyhemoglobin and the length of the light path, and the product ([oxyHb]+[deoxyHb]) of the concentration of the total hemoglobin and the length of the light path, respectively, optical brain function imaging devices use a near-infrared spectrometer (hereinafter abbreviated as NIRS), for example (see Patent Document 1).

FIG. 6 is a block diagram schematically showing an example of the structure of a conventional optical brain function imaging device.

The optical brain function imaging device (near-infrared spectrometer) 101 has a housing 6 in rectangular parallelepiped form. A light source for emitting light (light emitter) 102, a light source driving mechanism 4 for driving the light source 102, a light detector (light receiver) 103 for detecting information on the amount of received light Aₙ (λₘ), an A/D converter 5, a controller for light transmission and reception 21, a controller for analysis 22, and a memory 23 are provided in the housing 6, and at the same time, a holder 50 to be mounted on the head of a subject, N light transmitting probes 112 to be fixed to the holder 50, M light receiving probes 113 to be fixed to the holder 50, a display device 26 having a monitor screen 26a, and a keyboard (input device) 27 are provided on the outside of the housing 6.

The light source driving mechanism 4 drives the light source 102 through a drive signal inputted from the controller for light transmission and reception 21. The light source 102 consists of semiconductor lasers LD1, LD2 and LD3 for emitting near-infrared rays having three different wavelengths λ₁, λ₂ and λ₃, for example.

The light detector 103 detects near-infrared rays having respective wavelengths, and thus outputs light reception signals (information on the amounts of received light) A(λ₁), A(λ₂) and A(λ₃) to the controller for light transmission and reception 21 through the A/D converter 5, and a photomultiplier tube, for example, is used as the detector.

This near-infrared spectrometer 101 uses the holder 50 in order to make the N light transmitting probes 112 and the M light receiving probes 113 make close contact with the surface of the head of a subject in a predetermined arrangement. The holder 50 that is used is molded in a bowl shape so as to conform to the shape of the surface of a head, for example. FIG. 7 is a perspective diagram showing an example of the holder. (N+M) through holes (attachment portions) 51 are created in the holder 50 with a distance of 30 mm between them in rows and columns. The through holes 51 are in a cylindrical shape having a diameter of approximately 10 mm and a depth of approximately 5 mm.

FIGS. 8(a) to 8(c) are diagrams showing an example of a light transmitting probe (light receiving probe). FIG. 8(a) is a perspective diagram showing a light transmitting probe, FIG. 8(b) is a cross-sectional diagram showing a light transmitting probe, and FIG. 8(c) is a front diagram showing a light transmitting probe.

The light transmitting probe 112 has a housing 112a in a cylindrical shape having an outer diameter of approximately 10 mm so that the housing 112a can fit into a through hole 51. One end of a light transmitting optical fiber 130a in a tubular shape having a diameter of 2 mm is inserted into the housing 112a. As a result, the other end of the light transmitting optical fiber 130a can be connected to the light emitter 102 so that near-infrared rays that have entered through the one end of the light transmitting optical fiber 130a can pass through the light transmitting optical fiber 130a so as to emit through the other end of the light transmitting optical fiber 130a (the end of the light transmitting probe 112).

The light receiving probe 113 has a similar structure as the light transmitting probe 112, and thus, also has a housing 113a in a cylindrical shape having an outer diameter of approximately 10 mm so that the housing 113a can fit into a through hole 51. One end of a light transmitting optical fiber 140a in a tubular shape having a diameter of 2 mm is inserted into the housing 113a. As a result, the other end of the light receiving optical fiber 140a can be connected to the light detector 103 so that near-infrared rays that have entered through the one end of the light receiving optical fiber 140a (the end of the light receiving probe 113) can pass through the light receiving optical fiber 140a so as to emit through the other end of the light receiving optical fiber 140a.

Thus, the N light transmitting probes 112 and the M light receiving probes 113 are inserted into the through holes 51 in the holder 50 alternately in rows and columns. FIG. 9 is a diagram showing an example of the positional relationship between N light transmitting probes and the M light receiving probes. Here, the light transmitting probes 112 are shown as the round, white sections, and the light receiving probes 113 are shown as the round, black sections.

Here, different numbers (T1, T2 . . . Tn, R1, R2 . . . Rm) are allocated to the through holes 51 so that it can be perceived which light transmitting probes 112_{T1} to 112_{Tn} or light receiving probes 113_{R1} to 113_{Rm} have been inserted into which through holes 51 in the holder 50, and at the same time, different numbers (T1, T2 . . . Tn) are allocated to the light transmitting probes 112_{T1} to 112_{Tn}, and different numbers (R1, R2 . . . Rm) are allocated to the light receiving probes 113_{R1} to 113_{Rm}. As a result, the light transmitting probes 112_{T1} to 112_{Tn} and the light receiving probes 113_{R1} to 113_{Rm} are respectively inserted into the through holes 51 of the corresponding number.

With the positional relationship between the N light transmitting probes 112_{T1} to 112_{Tn} and the M light receiving probes 113_{R1} to 113_{Rm}, it is necessary to adjust the timing in which light is emitted from the light transmitting probes 112 and the timing in which light is received by the light receiving probes 113 so that one light receiving probe 113 receives only light emitted from one light transmitting probe 112 instead of simultaneously receiving light emitted from a number of light transmitting probes 112. In order to do this, the memory 23 stores a control table showing the timing in which the light source 102 emits light and the timing in which the light detector 103 detects light.

The controller for transmitting and receiving light 21, where such a control table is stored in the memory 23, outputs a drive signal for transmitting light to one light transmitting probe 112 to the light source 102, and at the same time detects a light reception signal (information on the amount of received light) received by a light receiving probe 113 by means of the light detector 103 during a predetermined period of time. As a result, information on the amount of received light Aₓ(λ₁), Aₓ(λ₂) and Aₓ(λ₃) concerning X portions to be measured is collected (x=1, 2 . . . X).

On the basis of the information on the amount of received light Aₓ(λ₁), Aₓ(λ₂) and Aₓ(λ₃) concerning X portions to be measured (x=1, 2 . . . X), the controller for analysis 22 uses the relational expressions (1), (2) and (3) to find the product [oxyHb] of the concentration of oxyhemoglobin and the length of the light path, the product [deoxyHb] of the concentration of deoxyhemoglobin and the length of the light path, and the product of the concentration of the total hemoglobin and the length of the light path ([oxyHb]+[deoxyHb]) from the intensity of light having the respective wavelengths (wavelength absorbed by oxyhemoglobin and wavelength absorbed by deoxyhemoglobin) that has passed through the portions to be measured.

### Prior Art Documents

### Patent Document

Patent Document 1: Japanese Unexamined Patent Publication 2006-109964

### Non-Patent Document

Non-Patent Document 1: Factors affecting the accuracy of near-infrared spectroscopy concentration calculations for focal changes in oxygenation parameters, Neurolmage 18, 865-879, 2003.

### SUMMARY OF THE INVENTION

### Problem to be Solved by the Invention

The light transmitting probes 112_{T1} to 112_{Tn} and the light receiving probes 113_{R1} to 113_{Rm} are fixed into the through holes 51 in the holder 50 after the holder 50 has been mounted on the head of a subject, and there is hair on the surface of the head, which makes it necessary for the tips of the light transmitting probes 112_{T1} to 112_{Tn} and the light receiving probes 113_{R1} to 113_{Rm} to make contact with the surface of the head and avoid the hair. Therefore, the task of pushing the hair aside is necessary when the light transmitting probes 112_{T1} to 112_{Tn} and the light receiving probes 113_{R1} to 113_{Rm} are attached.

Thus, the hair needs to be pushed aside when the light transmitting probes 112_{T1} to 112_{Tn} and the light receiving probes 113_{R1} to 113_{Rm} are fixed into the through holes 51 in the holder 50, which is very troublesome for the doctor and very stressful for the subject whose movement is restricted for a long period of time.

Furthermore, some subjects exercise everyday for rehabilitation, and in the case where the subject does this at home, it is very troublesome and takes a long time for a family to fix the light transmitting probes 112_{T1} to 112_{Tn} and the light receiving probes 113_{R1} to 113_{Rm} into the through holes 51 in the holder 50 on the head of the subject.

### Means for Solving Problem

The inventor carried out examinations on probes that could be placed on the head of a subject in a short period of time. With the above-described probes, it is necessary for the hair to be pushed aside when the tips of the probes are made to make contact with the surface of the head. Therefore, the inventor found it a good idea for the probes to push the hair aside when they are fixed to the holder. That is to say, the tips of the probes are made in a comb shape.

The light transmitting probe according to the present invention has: a housing for fixing the probe to a mounting portion of a holder to be mounted on a subject; a light emitter for emitting light placed in an end portion of the above-described housing; and a transmission channel, one end of which is connected to the light emitter and the other of which is connected to a controller, and the light transmitting probe irradiates the subject with light when fixed to the above-described holder, wherein the end portion of the above-described housing has a number of rod-shaped protrusions, the above-described light emitter is a number of light emitting elements which are respectively placed in an end portion of each protrusion, and the above-described transmission channel is a number of transmission channels which are respectively placed inside each protrusion.

The probe according to the present invention has a number of rod-shaped protrusions. That is to say, the end of the probe is in a comb shape. As a result, hair can be moved away simultaneously as the probe is inserted into the mounting portion of the holder.

### EFFECTS OF THE INVENTION

As described above, the light transmitting probe according to the present invention makes it possible for it to be placed on the head of a subject in a short period of time.

### Other Means for Solving the Problem and Effects Thereof

The light receiving probe according to the present invention has: a housing for fixing the probe to a mounting portion of a holder to be mounted on a subject; a light receiver for detecting light placed in an end portion of the above-described housing; and a transmission channel, one end of which is connected to the light receiver and the other of which is connected to a controller, and the light receiving probe receives light emitted from the subject when fixed to the above-described holder, wherein the end portion of the above-described housing has a number of rod-shaped protrusions, the above-described light receiver is a number of light receiving elements which are respectively placed in an end portion of each protrusion, and the above-described transmission channel is a number of transmission channels which are respectively placed inside each protrusion.

As described above, the light receiving probe according to the present invention makes it possible for it to be placed on the head of a subject in a short period of time.

In addition, the light transmitting and receiving probe according to the present invention has: a housing for fixing the probe to a mounting portion of a holder to be mounted on a subject; a light emitter for emitting light placed in an end portion of the above-described housing; a transmission channel, one end of which is connected to the light emitter and the other of which is connected to a controller; a light receiver for detecting light placed in an end portion of the above-described housing; and a transmission channel, one end of which is connected to the light receiver and the other of which is connected to a controller, and the light transmitting and receiving probe irradiates the subject with light, and at the same time receives light emitted from the subject when fixed to the above-described holder, wherein the end portion of the above-described housing has a number of rod-shaped protrusions, the above-described light emitter is a number of light emitting elements which are placed in an end portion of each protrusion, the above-described light receiver is a number of light receiving elements which are placed in an end portion of each protrusion, and the above-described transmission channel is a number of transmission channels which are respectively placed inside each protrusion.

As described above, the light transmitting and receiving probe according to the present invention makes it possible for it to be placed on the head of a subject in a short period of time.

Furthermore, the light measurement device according to the present invention has any of the above-described probes, a holder to be mounted on a subject, and a controller for controlling light transmission or reception for the above-described probes.

### BRIEF DESCRIPTION OF THE DRAWINGS

FIG. 1 is a block diagram schematically showing an example of the structure of the optical brain function imaging device according to one embodiment of the present invention;

FIGS. 2(a) to 2(c) are diagrams showing an example of a light transmitting probe;

FIGS. 3(a) to 3(c) are diagrams showing an example of a light receiving probe;

FIGS. 4(a) to 4(c) are diagrams showing an example of a light transmitting and receiving probe;

FIGS. 5(a) and 5(b) are diagrams showing the relationship between a portion to be measured and the distance (channel) between a light transmitting probe and a light receiving probe;

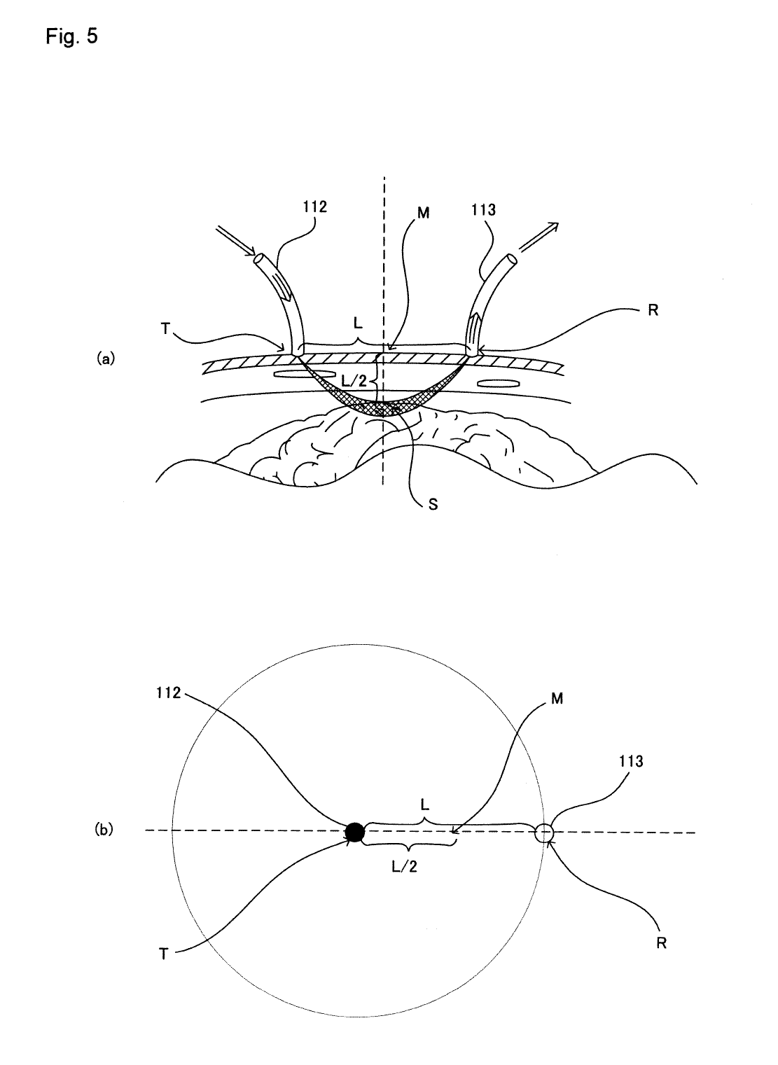

FIG. 6 is a block diagram schematically showing an example of the structure of a conventional optical brain function imaging device;

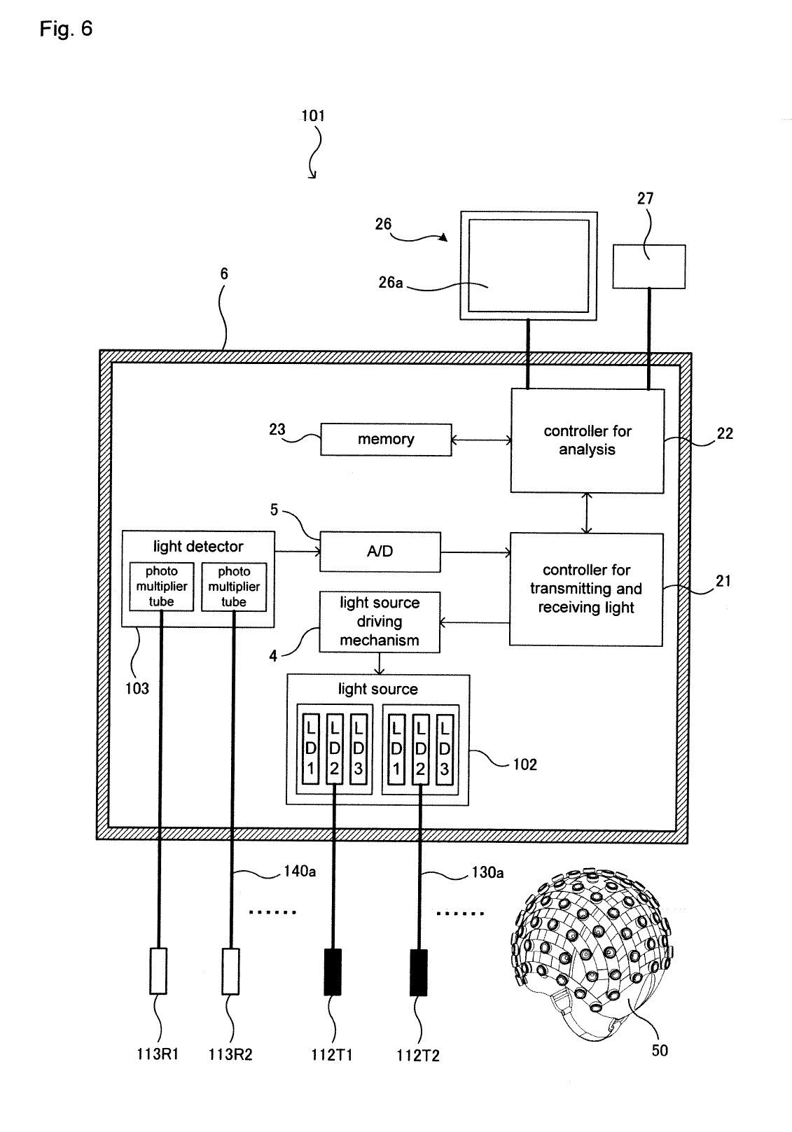

FIG. 7 is a diagram showing an example of the holder;

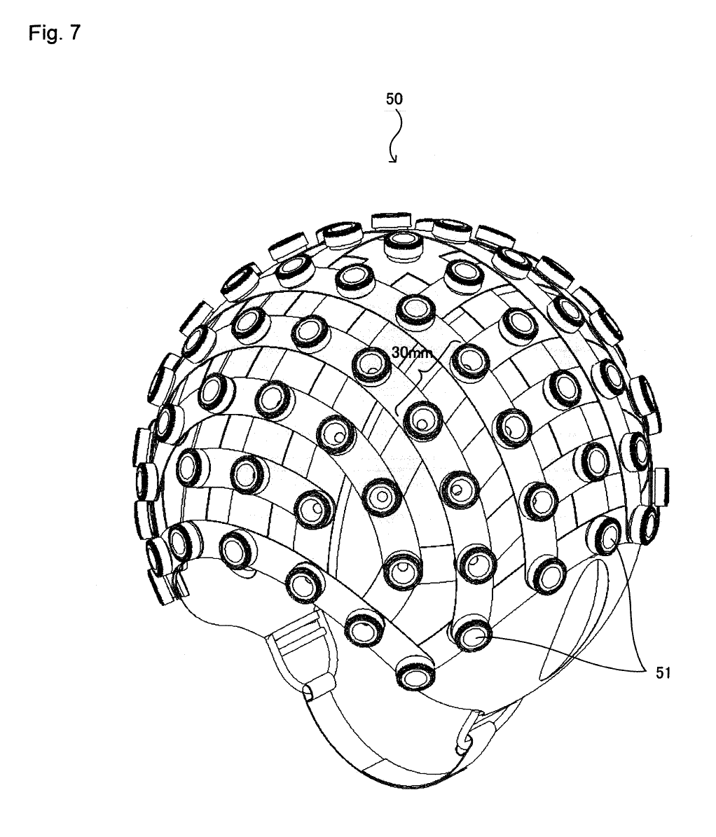

FIGS. 8(a) to 8(c) are diagrams showing an example of a light transmitting probe (light receiving probe); and

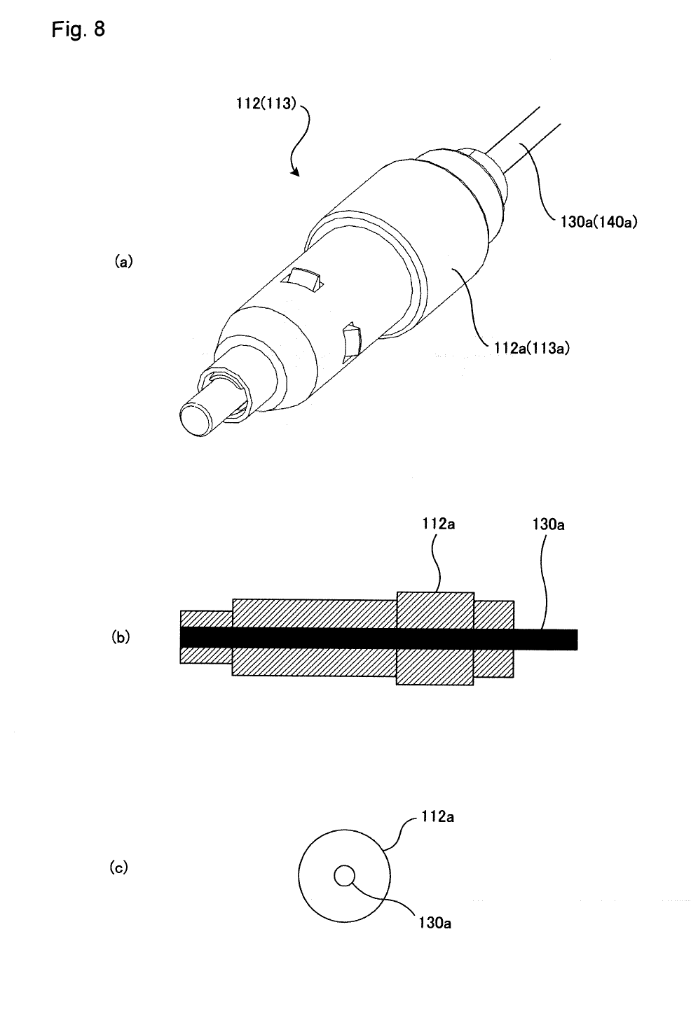

FIG. 9 is a diagram showing an example of the positional relationship between the N light transmitting probes and the M light receiving probes.

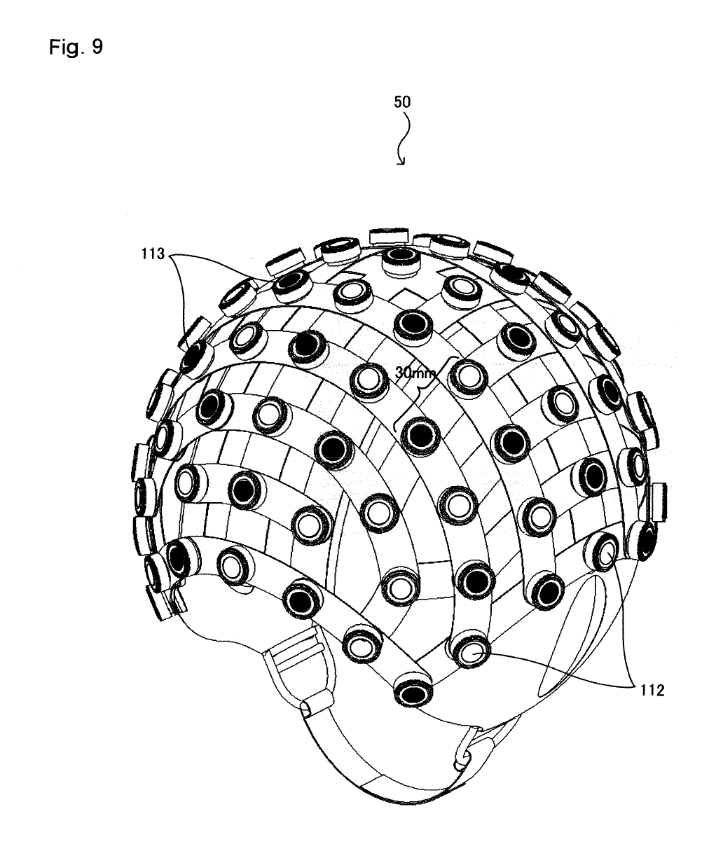

### BEST MODE FOR CARRYING OUT THE INVENTION

In the following, the embodiments according to the present invention are described in reference to the drawings. Here, the present invention is not limited to the below-described embodiments, and various modifications are naturally included as long as the gist of the present invention is not deviated from.

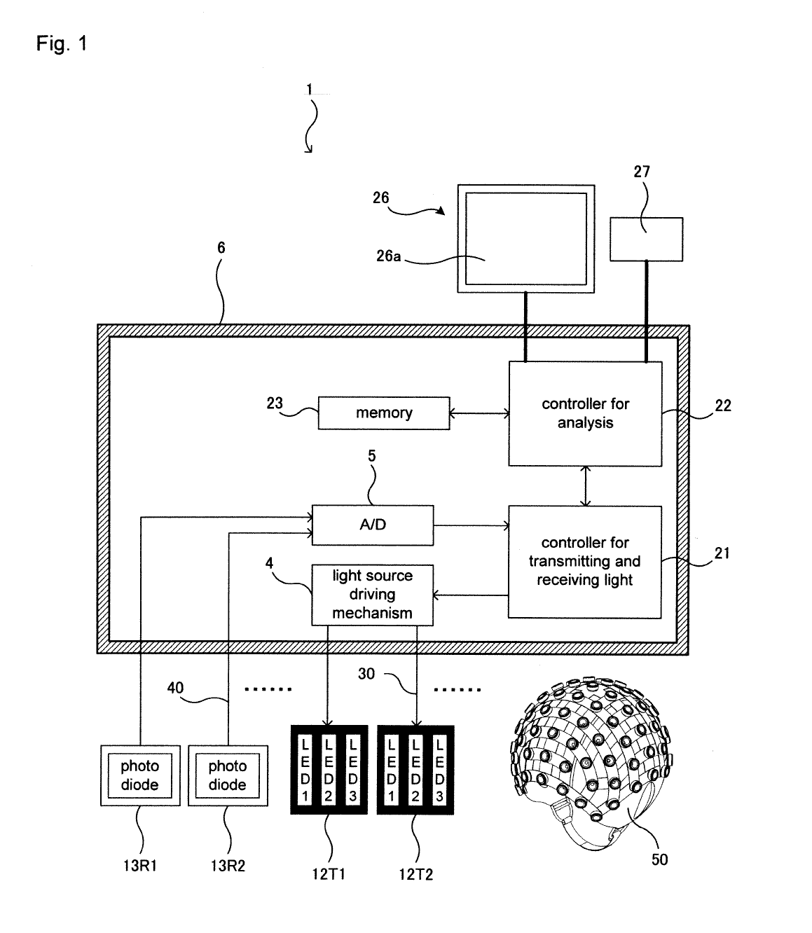

FIG. 1 is a block diagram schematically showing an example of the structure of the optical brain function imaging device according to one embodiment of the present invention. Here, the same symbols are attached to the same components as in the optical brain function imaging device 101.

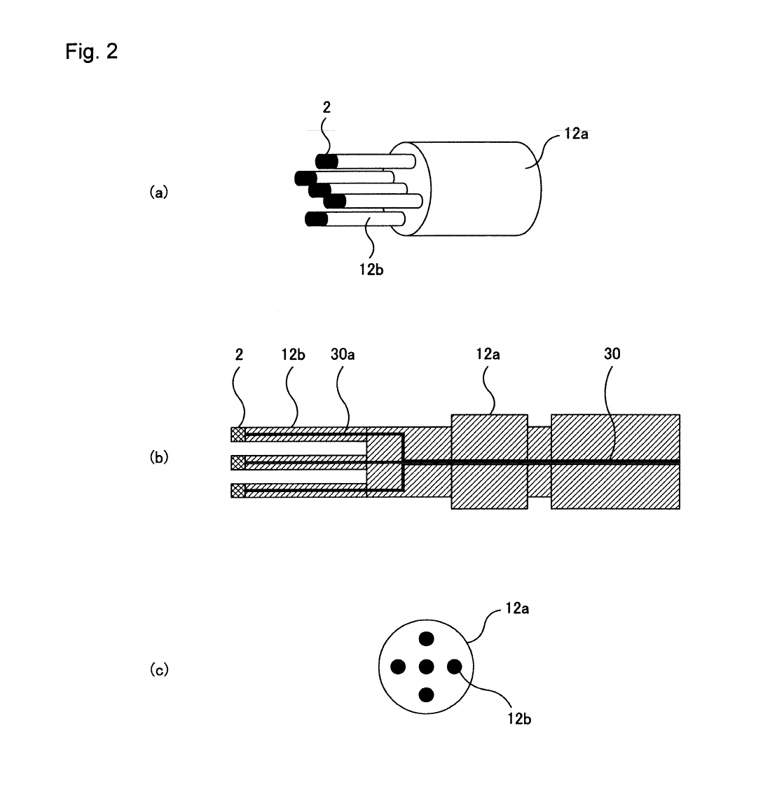

The optical brain function imaging device (near-infrared spectrometer) 1 has a housing 6 in rectangular parallelepiped form. A light source driving mechanism 4 for driving a light emitter 2 (see FIGS. 2(a) to 2(c)), an A/D converter 5, a controller for light transmission and reception 21, a controller for analysis 22, and a memory 23 are provided in the housing 6, and at the same time, a holder 50 to be mounted on the head of a subject, N light transmitting probes 12 to be fixed to the holder 50, M light receiving probes 13 to be fixed to the holder 50, a display device 26 having a monitor screen 26a, and a keyboard (input device) 27 are provided on the outside of the housing 6.

FIGS. 2(a) to 2(c) are diagrams showing an example of a light transmitting probe. FIG. 2(a) is a perspective diagram showing the light transmitting probe, FIG. 2(b) is a cross-sectional diagram showing the light transmitting probe, and FIG. 2(c) is a front diagram showing the light transmitting probe.

The light transmitting probe 12 has a housing 12a in cylindrical shape, and the housing 12a can fit into a through hole 51. Five protrusions 12b in columnar shape that run along the axis are formed in an end portion of the housing 12a. The diameter of the protrusions 12b is approximately 1 mm, and the length of the protrusions 12b is approximately 10 mm to 20 mm. Thus, as shown in FIG. 2(c), one protrusion 12b is located at the center of the light transmitting probe 12, and the other four protrusions 12b are arranged in a circle with equal intervals near the periphery portions of the light transmitting probe 12 as the end portion of the light transmitting probe 12 is viewed in the axial direction.

LEDs (light emitting diodes) 2 are respectively fixed to the end portion of each protrusion 12b. The LEDs 2 can emit near-infrared rays having three different wavelengths λ₁, λ₂ and λ₃, for example.

Each protrusion 12b has one end portion of a wire (transmission channel) 30a in tubular shape having a diameter of 1 mm inserted therein. In addition, the end portion of the wire 30a is connected to an LED 2. Furthermore, the other ends of the wires 30a are connected to each other, forming a wire 30 at one end, and the other end of the wire 30 is connected to the light source driving mechanism 4. As a result, the light source driving mechanism 4 can drive the LEDs 2 using a drive signal inputted from the controller for transmitting and receiving light 21.

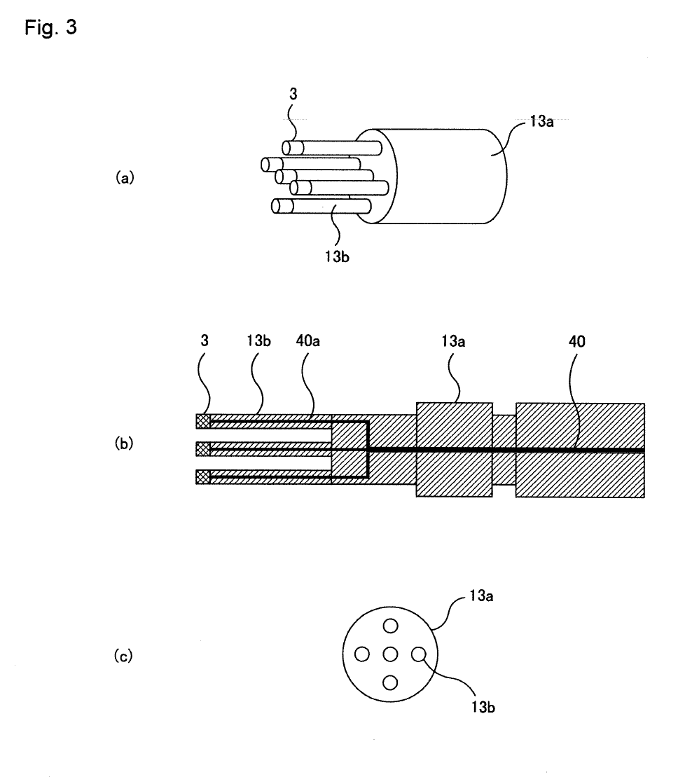

FIGS. 3(a) to 3(c) are diagrams showing an example of a light receiving probe. FIG. 3(a) is a perspective diagram showing the light receiving probe, FIG. 3(b) is a cross-sectional diagram showing the light receiving probe, and FIG. 3(c) is a front diagram showing the light receiving probe.

The light receiving probe 13 has a housing 13a in cylindrical shape, and the housing 13a can fit into a through hole 51. Five protrusions 13b in columnar shape that run along the axis are formed in an end portion of the housing 13a. The diameter of the protrusions 13b is approximately 1 mm, and the length of the protrusions 13b is approximately 10 mm to 20 mm. Thus, one protrusion 13b is located at the center of the light receiving probe 13, and the other four protrusions 13b are arranged in a circle with equal intervals near the periphery portions of the light receiving probe 13 as the end portion of the light receiving probe 13 is viewed in the axial direction.

Photodiodes (light receiving diodes) 3 are respectively fixed to the end portion of each protrusion 13b. The photodiodes 3 can detect near-infrared rays so as to output light reception signals (information on the amount of received light) A(λ₁), A(λ₂) and A(λ₃), respectively.

Each protrusion 13b has one end portion of a wire (transmission channel) 40a in tubular shape having a diameter of 1 mm inserted therein. In addition, the end portion of the wire 40a is connected to a photodiode 3. Furthermore, the other ends of the wires 40a are connected to each other, forming a wire 40 at one end, and the other end of the wire 40 is connected to the controller for transmitting and receiving light 21 through the A/D converter 5. As a result, the photodiodes 3 can output a light reception signal (information on the amount of received light) A(λ₁), A(λ₂) and A(λ₃) to the controller for transmitting and receiving light 21 through the A/D converter 5.

The light transmitting probes 12_{T1} to 12_{Tn} and the light receiving probes 13_{R1} to 13_{Rm} are fixed into the through holes 51 in the holder 50 after the holder 50 has been mounted on the head of a subject. Even if there is hair on the surface of the head of the subject, the hair is pushed aside when the end portions of the light transmitting probes 12_{T1} to 12_{Tn} and the light receiving probes 13_{R1} to 13_{Rm} are inserted into the through holes 51, and therefore, the holder 50 can be mounted on the head of the subject in a short period of time.

### Other Embodiments

Though it has been shown that the above-described optical brain function imaging device 1 has such a structure that N light transmitting probes 12 and M light receiving probes 13 are used, the structure may use (N+M) light transmitting and receiving probes 14.

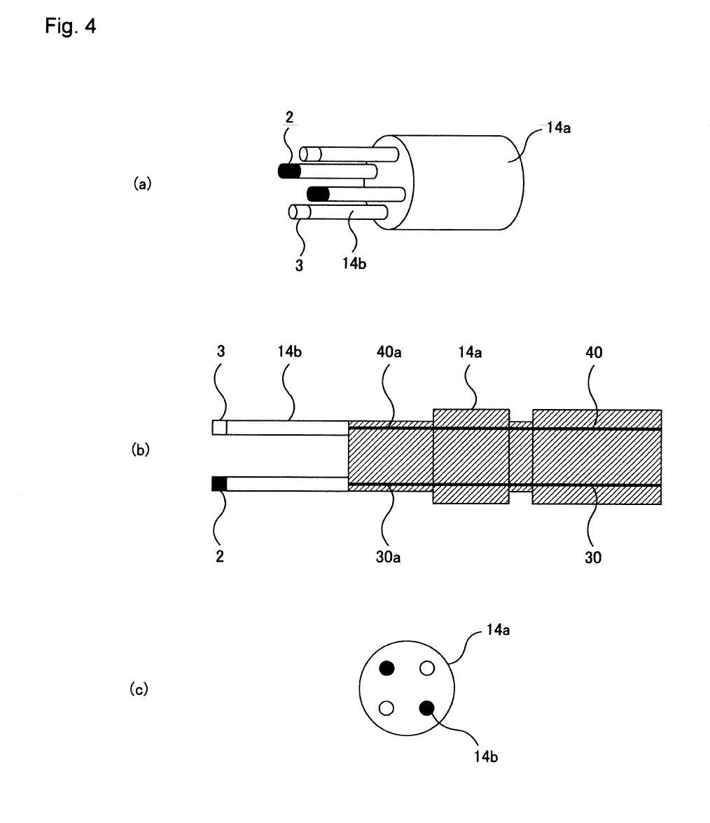

FIGS. 4(a) to 4(c) are diagrams showing an example of a light transmitting and receiving probe. FIG. 4(a) is a perspective diagram showing the light transmitting and receiving probe, FIG. 4(b) is a cross-sectional diagram showing the light transmitting and receiving probe, and FIG. 4(c) is a front diagram showing the light transmitting and receiving probe.

The light transmitting and receiving probe 14 has a housing 14a in cylindrical shape, and the housing 14a can fit into a through hole 51. Four protrusions 14b in columnar shape that run along the axis are formed in an end portion of the housing 14a. The diameter of the protrusions 14b is approximately 1 mm, and the length of the protrusions 14b is approximately 10 mm to 20 mm. Thus, the four protrusions 14b are arranged in a circle with equal intervals near the periphery portions of the light transmitting and receiving probe 14 as the end portion of the light transmitting and receiving probe 14 is viewed in the axial direction.

LEDs (light emitting elements) 2 are respectively fixed to the end portions of the first and third protrusions 14b. The first and third protrusions 14b have one end portion of a wire (transmission channel) 30a in a tubular shape having a diameter of 1 mm inserted therein. In addition, the end portion of the wire 30a is connected to an LED 2. Furthermore, the other end portions of the wires 30a are connected to each other, forming a wire 30 at one end, and the other end of the wire 30 is connected to the light source driving mechanism 4. As a result, the light source driving mechanism 4 can drive the LEDs 2 using a drive signal inputted from the controller for transmitting and receiving light 21.

Photodiodes (light receiving elements) 3 are respectively fixed to end portions of the second and fourth protrusions 14b. The second and fourth protrusions 14b have one end portion of a wire (transmission channel) 40a in a tubular shape having a diameter of 1 mm inserted therein. In addition, the end portion of the wire 40a is connected to a photodiode 3. Furthermore, the other end portions of the wires 40a are connected to each other, forming a wire 40 at one end, and the other end of the wire 40 is connected to the controller for transmitting and receiving light 21 through the A/D converter 5. As a result, the photodiodes 3 can output a light reception signal (information on the amount of received light) A(λ₁), A(λ₂) and A(λ₃) to the controller for transmitting and receiving light 21 through the A/D converter.

### INDUSTRIAL APPLICABILITY

The present invention can be applied to an optical brain function imaging device for measuring brain activity noninvasively.

### Explanation of Symbols

1: optical brain function imaging device (light measurement device)
2: light emitting element (light emitter)
3: light receiving element (light detector)
12: light transmitting probe
12a: housing
12b: protrusion
21: controller for transmitting and receiving light
30: wire (transmission channel)
30a: wire (transmission channel)
50: holder
51: through hole (attachment portion)

1. A light transmitting probe, comprising:
a housing for fixing the probe to a mounting portion of a holder to be mounted on a subject;
a light emitter for emitting light placed in an end portion of said housing; and
a transmission channel, one end of which is connected to the light emitter and the other of which is connected to a controller,
the light transmitting probe irradiating the subject with light when fixed to said holder and being characterized in that
the end portion of said housing has a number of rod-shaped protrusions,
said light emitter is a number of light emitting elements which are respectively placed in an end portion of each protrusion, and
said transmission channel is a number of transmission channels which are respectively placed inside each protrusion.

2. A light receiving probe, comprising:
a housing for fixing the probe to a mounting portion of a holder to be mounted on a subject;
a light receiver for detecting light placed in an end portion of said housing; and
a transmission channel, one end of which is connected to the light receiver and the other of which is connected to a controller,
the light receiving probe receiving light emitted from the subject when fixed to said holder and being characterized in that
the end portion of said housing has a number of rod-shaped protrusions,
said light receiver is a number of light receiving elements which are respectively placed in an end portion of each protrusion, and
said transmission channel is a number of transmission channels which are respectively placed inside each protrusion.

3. A light transmitting and receiving probe, comprising:
a housing for fixing the probe to a mounting portion of a holder to be mounted on a subject;
a light emitter for emitting light placed in an end portion of said housing;
a transmission channel, one end of which is connected to the light emitter and the other of which is connected to a controller;
a light receiver for detecting light placed in an end portion of said housing; and
a transmission channel, one end of which is connected to the light receiver and the other of which is connected to the controller,
the light transmitting and receiving probe irradiating the subject with light and receiving light emitted from the subject when fixed to said holder and being characterized in that
the end portion of said housing has a number of rod-shaped protrusions,
said light emitter is a number of light emitting elements which are placed in an end portion of each protrusion,
said light receiver is a number of light receiving elements which are placed in an end portion of each protrusion, and
said transmission channel is a number of transmission channels which are respectively placed inside each protrusion.

4. A light measurement device, characterized by comprising:
the probe according to any of claim 1;
a holder to be mounted on a subject; and
a controller for controlling light transmission or light reception for said probe.

5. A light measurement device, characterized by comprising:
the probe according to any of claim 2;
a holder to be mounted on a subject; and
a controller for controlling light transmission or light reception for said probe.

6. A light measurement device, characterized by comprising:
the probe according to any of claim 3;
a holder to be mounted on a subject; and
a controller for controlling light transmission or light reception for said probe.
